(19) United States
(12) Patent Application Publication
(10) Pub. No.: US 2013/0243920 A1
(43) Pub. Date: Sep. 19, 2013

(54) Teeth color maintenance composition for addition into beverages

(71) Applicant: Dino Shahrier Javaheri, Danville, CA (US)

(72) Inventor: Dino Shahrier Javaheri, Danville, CA (US)

(21) Appl. No.: 13/423,611

(22) Filed: Mar. 19, 2012

(51) Int. Cl. A23L 2/52; C01B 15/01; C01D 7/00; C01B 11/02; C01B 15/12; C07C 275/02

(52) U.S. Cl. 426/442; 423/277; 564/63; 423/415.2; 423/477; 423/584

(57) ABSTRACT

Disclosed is a process and composition for teeth bleaching and color maintenance made up of a solution added to beverages (coffee, tea, wine, sodas). The method and composition offers and option to start breaking down stains prior to penetration in o the tooth enamel. The purpose of this invention is to provide a way to eliminate or reduce the staining effect from beverages such as coffee, tea, wine, juices and sodas. The mixture will include an oxygen releasing agent which may include infusion of oxygen gas, and/or solutions of sodium perborate, carbamide peroxide, chlorine dioxide, or hydrogen peroxide. The mixture is dispensed into drinks that can stain teeth.

### FIELD OF THE INVENTION

This invention relates to the field of tooth whitening and color maintained using oxygen release and a hydrophilic food additive introduced into beverages that discolor teeth.

### BACKGROUND OF THE INVENTION

Teeth whiteners, also known as teeth bleaching agents, are available to consumers in many different forms. There are systems that are available only through a dentist, involving high strength peroxide solutions. There are also application protocols involving peroxide gels dispends in trays or adhesive trays or adhesive strips. All of these protocols involve removing the stains once they are embedded into the teeth. Below is a list of references that may or may not be material to the prosecution and may or may not be prior art.

| | | Name of Patentee or |
|---|---|---|
| Document Number | Publication Date | Applicant of Cited Document |
| U.S. Pat. No. 4,990,089 | February 1991 | Munro |
| U.S. Pat. No. 5,076,791 | December 1991 | Madray, Jr. |
| U.S. Pat. No. 5,234,342 | August 1993 | Fischer |
| U.S. Pat. No. 5,376,006 | December 1994 | Fischer |
| U.S. Pat. No. 5,409,631 | April 1995 | Fischer |
| U.S. Pat. No. 5,718,886 | February 1998 | Pellico |
| U.S. Pat. No. 6,290,934 | September 2001 | Kramer et al. |
| U.S. Pat. No. 6,365,134 | April 2002 | Orlowski et al. |
| U.S. Pat. No. 6,447,757 | September 2002 | Orlowski et al. |

### SUMMARY OF THE INVENTION

The present invention is designed to overcome the deficiencies of the methods discussed above. It is the object of the present invention is to provide a process and composition to eliminate or reduce the staining effect from beverages.

### DETAILED DESCRIPTION OF THE INVENTION

The invention relates to a new approach to prevent teeth from stains in beverages such coffee, tea, wine, sodas and juices. This invention is a maintenance system to preserve the color of teeth. The scope of the invention presently disclosed extends beyond the embodiments presented herein.

In preferred embodiments, the composition is a liquid, table or powder that releases oxygen when introduced into a beverage. The oxygen releasing agent (sodium perborate, carbamide peroxide, chlorine dioxide or hydrogen peroxide) is preferably present at a concentration from about 1%-10%. Water is present at 90% to 99%. Oxygen gas may also be infused into the solution. The invention is added into beverages, the oxygen release leads to a breakdown of teeth staining molecules. By breaking down the discoloring component of the beverage prior to its contact with teeth, teeth staining is reduced.

1. A method for whitening teeth comprising adding an agent that releases oxygen to a food.

2. The method of claim 1, the agent being a hydrophilic additive formed to have a pH of about 4 to 10.

3. The method of claim 1, the agent being in one of in liquid, tablet, or powder form.

4. (canceled)

5. The method of claim 1 as the agent being sodium perborate, carbamide peroxide, sodium percarbonate, chlorine dioxide or hydrogen peroxide.

6. The composition of claim 1, comprises 1-10% by weight of the oxygen releasing agent.

7. The composition of claim 1, comprises 90-99% by weight water.

8. The composition of claim 1, further comprising of one or more additives including at least food grade colorants, flavors, or fragrances.

9. The method of claim 1, the food being a beverage that stains teeth.

10. (canceled)

11. The method of claim 1 the agent being sodium perborate.

12. The method of claim 1 the agent being carbamide peroxide.

13. The method of claim 1 the agent being sodium percarbonate.

14. The method of claim 1 the agent being chlorine dioxide.

15. The method of claim 1 the agent being hydrogen peroxide.

16. The method of claim 1 the agent being sodium perborate, carbamide peroxide, sodium percarbonate, chlorine dioxide and hydrogen peroxide.

17. The method of claim 1 the food being a beverage, the method further comprising stirring the food for 5 seconds with a mixer.

18. The method of claim 1 the food being coffee.

19. The method of claim 1, the food being tea.

20. The method of claim 1, the food being wine.

21. The method of claim 1, the food being juice.

22. The method of claim 1, the food being soda.

23. A composition of matter comprising: sodium perborate, carbamide peroxide, sodium percarbonate, chlorine dioxide or hydrogen peroxide in an amount effective for reducing teeth discoloration when mixed in a tooth discoloring food.
